(19) United States
(12) Patent Application Publication
(10) Pub. No.: US 2016/0106595 A1
(43) Pub. Date: Apr. 21, 2016

(54) USER-CONFIGURABLE PRECUT KINESIOLOGY TAPE STRIP

(71) Applicant: SPIDERTECH INC., Toronto (CA)

(72) Inventors: Ray Arbesman, Toronto (CA); Kevin Jardine, Toronto (CA)

(73) Assignee: SPIDERTECH INC., Toronto, ON (CA)

(21) Appl. No.: 14/894,123

(22) PCT Filed: May 28, 2014
(86) PCT No.: PCT/CA2014/000463

§ 371 Date: Nov. 25, 2015

(30) Foreign Application Priority Data
May 29, 2013 (CA) 2,817,231

(51) Int. Cl. A61F 13/02; A61F 13/00

(52) U.S. Cl. CPC A61F 13/0259; A61F 13/00059; A61F 13/00085; A61F 2013/00119; A61F 2013/0028

(57) ABSTRACT

A user-configurable precut kinesiology tape strip is provided for application by a user to a patient. Each end of the strip lobed, having troughs between the lobes. The lobes are in pairs opposite each other. The backing paper is marked with grid lines connecting troughs between the lobes. The grid lines allow the user to selectively cut the strip to form it into any of a number of possible configurations prior to removing the backing paper and applying the strip to a patient's body. A method of configuring such strips is also provided.

### RELATED APPLICATION

This application claims the benefit of priority to Canadian application No. 2,817,231 filed May 29, 2013, the contents of which are incorporated herein by reference in their entirety.

### FIELD OF THE INVENTION

The invention relates to kinesiology tapes, and more particularly relates to precut strips of kinesiology tape for application to patients.

### BACKGROUND OF THE INVENTION

Kinesiology tape is a form of high-stretch adhesive support tape that acts as an assist to weak musculature. The stretch in the tape allows a mild degree of tension to be placed across the supported body part, in effect acting as an auxiliary muscle.

Kinesiology tape can also be used for pain therapy in acute situations. The tape lifts the skin providing a stimulus to the fascia and/or muscular tissue, stimulates blood and lymphatic flow, alters the forces over the affected joint, effects muscle inhibition and facilitation, and stimulates muscle and skin receptors such as mechanoreceptors, nociceptors, exteroceptive receptors, and cutaneous proprioceptive afferents.

The adhesive aspect of the tape is critical to producing these effects. The tape sticks directly to the body. The adhesive is strong enough that opposite ends of a length of tape applied to the body will remain adhered even when the tape length is under tension and the body part is in regular active use.

Kinesiology tape in roll form has been used for many years. Kinesiology tape is used by cutting strips from a roll that are then further cut (all by hand) to form individualized therapeutic applications. This cutting and shaping is time-consuming. Another drawback of existing kinesiology tape formats is that the roll product is frequently only available in narrower widths (less than 3 inches). While a narrow width provides a convenient size for packaging on a roll, the size is not necessarily conducive to effective coverage for support of many body parts. The doctor applying the tape may have to cut and apply multiple pieces of tape to achieve a desired effect or coverage. This complicates the application process and increases the time commitment.

More recently, precut kinesiology tape applications have become known. One example is taught and described in CA 2,578,927 (Arbesman et al.), titled “Precut Adhesive Body Support Articles and Support System”. Such precut applications have found enormous popularity in the mass market due to their speed of application and ease of use.

The trend in precuts is toward greater levels of user-friendliness. Accordingly, the available precut applications are each directed to a different body part or end-shape. The user does not need to do any cutting or shaping to obtain a usable tape brace ready for application. This makes the precut applications particularly suited for the direct-to-consumer market, as they are essentially “fool-proof.”

However, in a professional practice, such precuts may be perceived as excessively limiting. The professional user may desire a greater degree of flexibility than is presently available with precuts. However, the alternative of cutting from a tape roll may be time-prohibitive and hassle-prone. Further, rolled tape has limited width and may not be suitable for larger applications. Further, each cut piece must have its ends hand-rounded each time. This hand-cutting of the ends is time-consuming, and a raw edge may be left that leads to undesirable edge lift or fouling. The hand-cut strips from a roll of tape are also highly non-standardized, being different each time they are cut, even by the same professional for the same patient. There is also a difficulty in obtaining detailed and long cuts (e.g. lymphatic cuts) consistently and evenly.

Certain rolled tape strips may be useful in very basic applications, however, they too have limitations. The precut center-line(s) on these rolled tape strips can make them unsuitable for applications other than basic “I”, “Y” and “X” shapes. Further, they are limited in width and length, and therefore, may be unsuitable for larger applications. The precut center-line(s) can also open where unwanted, and therefore, cause problems in application.

In general, although there are many highly specialized forms of precuts available, it would be desirable to have a hybrid option that preserves some of the customizability of rolled tape, while obtaining certain benefits of precuts.

### SUMMARY OF THE INVENTION

According to a first aspect of the invention, a user-configurable precut kinesiology tape strip is provided for application by a user to a patient. A precut strip of kinesiology tape is provided that has a first end and a second end. Each of the ends has a plurality of lobes. The lobes are disposed opposite each other in pairs. The lobes on each end are generally adjacent to each other with troughs in between. A backing paper is provided over an adhesive side of the kinesiology tape strip. The paper is marked with a first set of longitudinal grid lines connecting opposing troughs from one end of the strip to the other. The longitudinal grid lines correspond to uncut portions of the strip and backing paper. The longitudinal grid lines permit selective cutting of the kinesiology tape strip along one or more of the longitudinal grid lines to form the strip into any one of a number of possible configurations prior to removal of the backing paper and application to a patient's body.

The strip may also include latitudinal grid lines marked at predetermined intervals on the backing paper. Such latitudinal grid lines may be straight across, or may have lobes and troughs matching the lobes and troughs of the nearest end of the strip. A second set of longitudinal grid lines may also be marked on the tape strip opposite the first set of longitudinal grid lines on the backing paper.

The lobes on the strip may be generally rounded in shape. The troughs may be generally V-shaped or notch-shaped. The troughs preferably represent spaces between the lobes.

Various dimensions are possible. However, it is preferred that the strip is at least approximately 10 inches in length, and at least approximately 3 inches wide. Each end preferably has at least approximately 3 lobes.

According to a second aspect of the invention, a method is provided for configuring a precut kinesiology tape strip for application of the strip by a user to a patient. The strip has a first end and a second end. Each end has a plurality of lobes. The lobes of opposite ends are disposed opposite each other in pairs. The lobes on each end are generally adjacent to each other with troughs in between. The strip has a backing paper over an adhesive side of the kinesiology tape strip. At least one of the backing paper and the strip is marked with a set of longitudinal grid lines connecting opposing troughs from one end of the strip to the other. The longitudinal grid lines correspond to uncut portions of the strip and backing paper. To configure the tape strip, the user selectively cuts along at least a portion of one or more of the longitudinal grid lines, each cut beginning at a trough. The cut(s) leave at least one anchor portion on the strip. This at least one anchor portion corresponds with a generally uncut portion of the strip that is selected by the user for first application to the patient. The at least one anchor portion can be selectively positioned anywhere along the strip, according to the preference of the user. The cut(s) also create a plurality of finger portions by virtue of the cut gridlines. The finger portions represent portions of the strip selected by the user for second and subsequent application to the patient following the application of the anchor portion.

When the strip further comprises latitudinal grid lines marked at predetermined intervals along either or both of the strip or the backing paper, the cutting step further includes selectively cutting along at least one of the vertical grid lines.

When the strip includes latitudinal grid lines having lobes and troughs matching the lobes and troughs of the nearest end of the strip, the cutting step further includes selectively cutting along at least one of the latitudinal grid lines to follow the lobes and troughs.

Being a user-configurable item, various cutting patterns and orders of operation are possible. Preferably, at least a portion of the longitudinal grid lines are cut prior to cutting at least a portion of the latitudinal grid lines. The cutting step may include cutting only a portion of the latitudinal grid lines corresponding to a finger portion of the strip to shorten or remove the finger portion. The cutting step may include cutting longitudinal grid lines parallel to each other to form a finger portion that is one grid line in width. The cutting step may include cutting longitudinal grid lines spaced apart by one or more longitudinal grid lines to form a finger portion that is more than one grid line in width. The cutting step may include forming cuts of different lengths. Alternatively, the cutting step may include forming multiple cuts of the same length. The cutting step may include cutting portions of the grid lines generally opposite each other or offset from each other at opposite ends.

### BRIEF DESCRIPTION OF THE DRAWINGS

FIG. 1 shows a first side of a user-configurable precut kinesiology tape strip, according to a preferred embodiment.

FIGS. 2A-2B show two embodiments of possible grid lines on the backing paper opposite the first side of the tape strip in FIG. 1.

FIGS. 3-8 are sample user configurations of the tape strip applied to a patient, showing portions of the tape that had previously been cut to form anchor portions and finger portions.

FIG. 9 shows a first stage of cutting a user-configurable precut kinesiology tape strip.

FIG. 10 shows a second stage wherein a user-configurable precut kinesiology tape strip has been cut to form anchor and finger portions ready for application to a patient.

### DETAILED DESCRIPTION

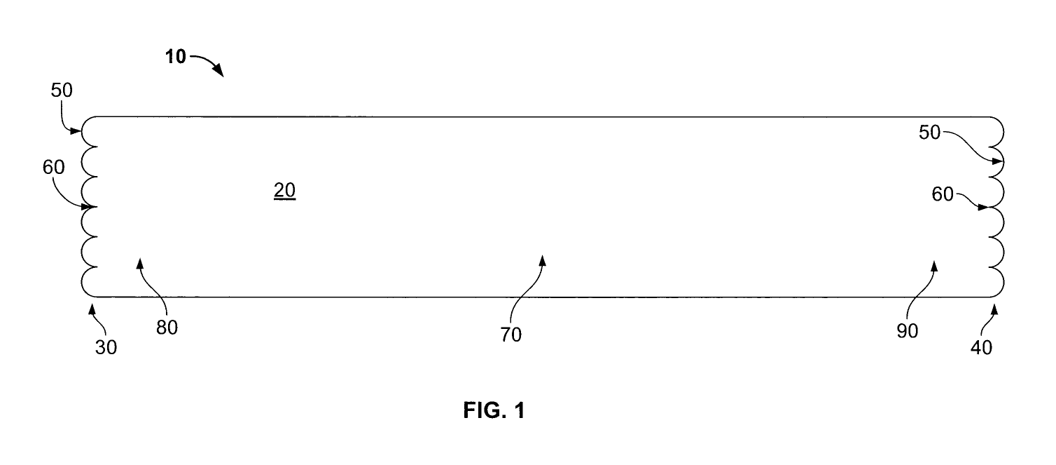

The user-configurable precut kinesiology tape strip 10 is shown in FIG. 1. The strip is made up of a strip of kinesiology tape having a high-elasticity cloth base to which is applied a pressure-sensitive adhesive. One side is a cloth side 30. On the adhesive side (not shown), a backing paper 100 is applied to prevent curling of the tape, and drying or fouling of the adhesive prior to use. The strip is both longer and wider than normal rolled kinesiology tapes. The cloth is preferably a woven fabric of natural or synthetic fibres with a high longitudinal elasticity. The adhesive is preferably a pressure-sensitive non-latex adhesive (e.g. a polyacrylic adhesive).

The strip has first 30 and second 40 ends. Each end has a preferably scalloped pattern (having lobes 50 separated by troughs 60, as shown in FIG. 1). The strip in this form can be thought of as having end regions 80, 90 and a central region 70.

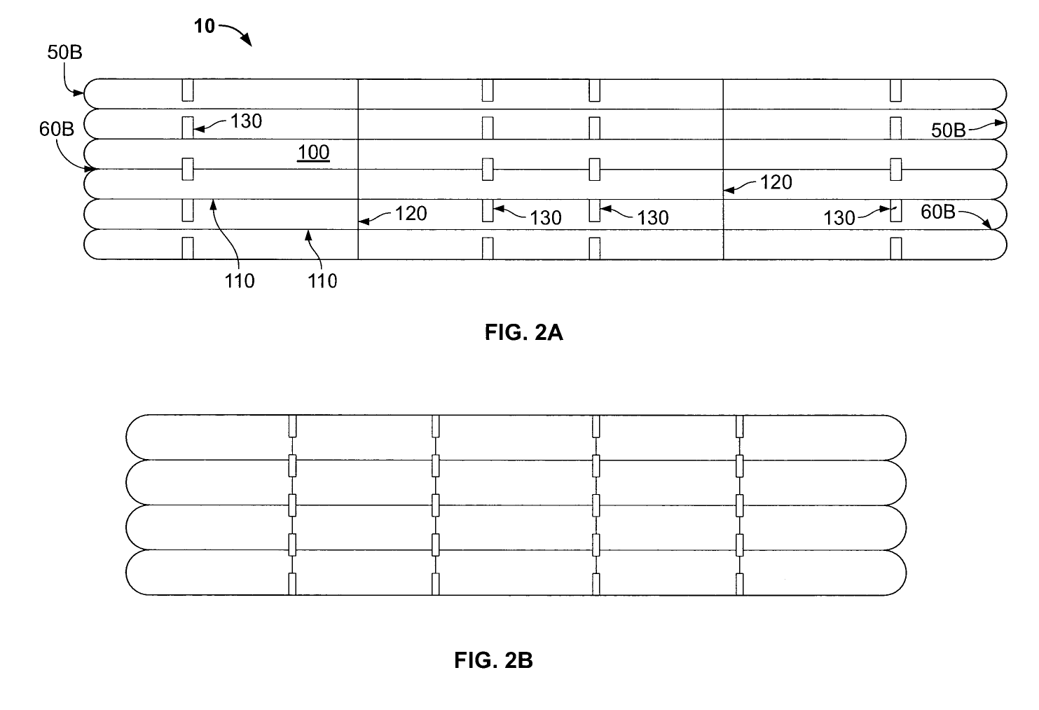

Two different layouts of backing paper 100 are shown in FIGS. 2A-2B. These represent two possible sizes of tape strip, and possible configurations of grid lines on the backing paper. Various layouts are possible. As shown in these figures, the backing paper 100 has printed on it a number of grid lines. Longitudinal grid lines 110 extend between (and preferably join) the troughs 60 from one end of the strip to the other. Latitudinal (or transverse) grid lines 120 may also run across the width of the tape strip. The grid lines may also (or in the alternative) be printed on the tape strip itself. The backing paper and/or the strip may also contain other indicia and cuttable regions (e.g. areas cuttable into windows, ports or slots in the strip). Preferably, the strip is integral and uncut over its length when sold. This integrity allows it to be applied in a whole (uncut) form (as shown in FIG. 4), and allows maximum flexibility in the placement of cuts.

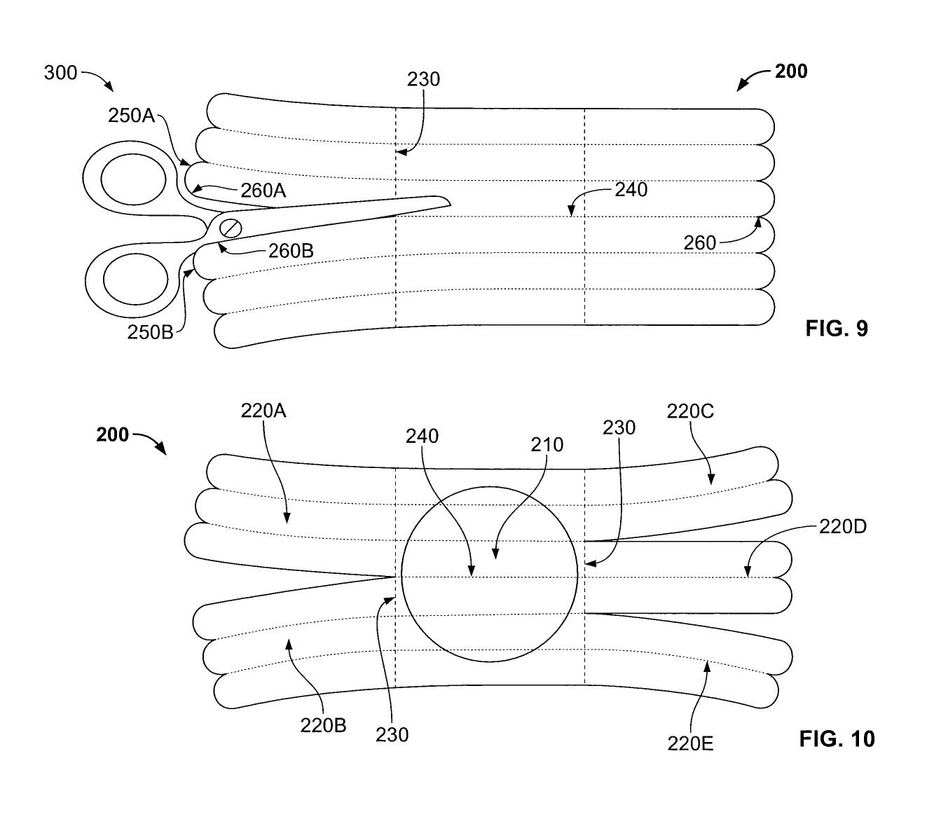

The grid lines 110, 120 permit cutting to shape the strip. As shown in FIGS. 9 and 10, to cut the configurable strip 200, a cut may be started from one of the troughs 260. The user may cut along the longitudinal grid line 240 as shown, separating the trough 260 to form cut edges 260A, 260B, until a latitudinal grid line position 230 is reached, or some other desired stopping point. Typically, the user will not cut all the way across the strip, but will stop at some point along the strip. The uncut portion(s) may form an anchoring portion (generally, shown here as the region notionally designated 210) (the anchoring portion is generally the area first applied to the patient), and the cut portions may form finger portions (here, 220A-E) extending outward from the notional anchoring portion 210 (these finger portions 220A-E are applied after the anchoring portion 210 to the patient). Note that finger portions may be formed in the latitudinal direction or the longitudinal direction (or a combination).

The grid lines allow the user to plan in advance where the strip is to be cut and enables the design to be formed symmetrically if desired or otherwise evenly and with intent (rather than haphazardly with a loose idea in mind, as tape rolls are cut at present). Unlike other strips which have a predetermined anchoring portion location, the strip can also be configured to have the anchoring portion disposed anywhere along the strip (not necessarily at one of the end regions 80, 90, or the central region 70). Therefore, the strips promote both creativity and repeatable standardization for applications.

The grid lines also facilitate documenting how the strip was cut for an individual patient so that repeat therapy can be practiced consistently and with ease.

The grid lines also facilitate cutting complicated fan shapes as straight and consistent finger cuts can be made even over a long length.

The latitudinal grid lines may also enable scaling the strip down by enabling trimming the finger portions to a desired length. The finger portions can also be removed entirely to form an enlarged window or slot. The latitudinal grid lines may be straight across (as shown) or may echo the scalloped/lobed pattern of the ends.

Scissors 300 may be provided with a kit of the configurable tape strips, or may be available separately. Preferably, for use in configuring the tape strip, fabric-type scissors or medical scissors are used, and the cutting edges may be provided with a coating to resist adhesive sticking.

Some of the grid lines of the backing paper may also be partially or fully perforated, scored or weakened (e.g. lines 130 shown in bold dashed lines in FIGS. 2A-B), to allow the backing paper to be separated in parts (as is known in the art). Cracking open or folding the perforated areas to separate portions of the backing paper, the backing paper is removed in sections and the now-configured tape strip 200 is positioned on the patient. The user applies the anchoring portion 210 to the patient, and then the finger portions 220A-E. The backing paper sections are removed in stages to allow each of these portions to be applied in stages. Premature removal of the backing paper results in twisting and tangling of the cut portions of the strip. The strip is laid on the patient without extending around the patient's body at any point. Further, the strip is applied (preferably in stages) so that a substantially continuous adhesive contact is made with the patient's skin over the length and width of the strip.

The dimensions of the strip are preferably at least 10 inches in length, and at least 3 inches in width. The number of lobes on each end is preferably at least 3. It may be desirable in certain embodiments to provide an even number of lobes so that evenly divided and symmetrical finger portions may be formed. However, symmetry and even-numbered lobes are not necessary. Further, it will be appreciated that the lines need not necessarily run parallel to (or across) the backing paper at right angles, but may be provided according to another scheme (e.g. windows of any shape may be outlined, and these may not necessarily be joined by grid lines to the ends or edges of the strip). Further, the adhesive may be patterned to follow some or all of the grid lines (e.g. to prevent the backing paper from removing prematurely from the adhesive of the strip as the user cuts the grid lines).

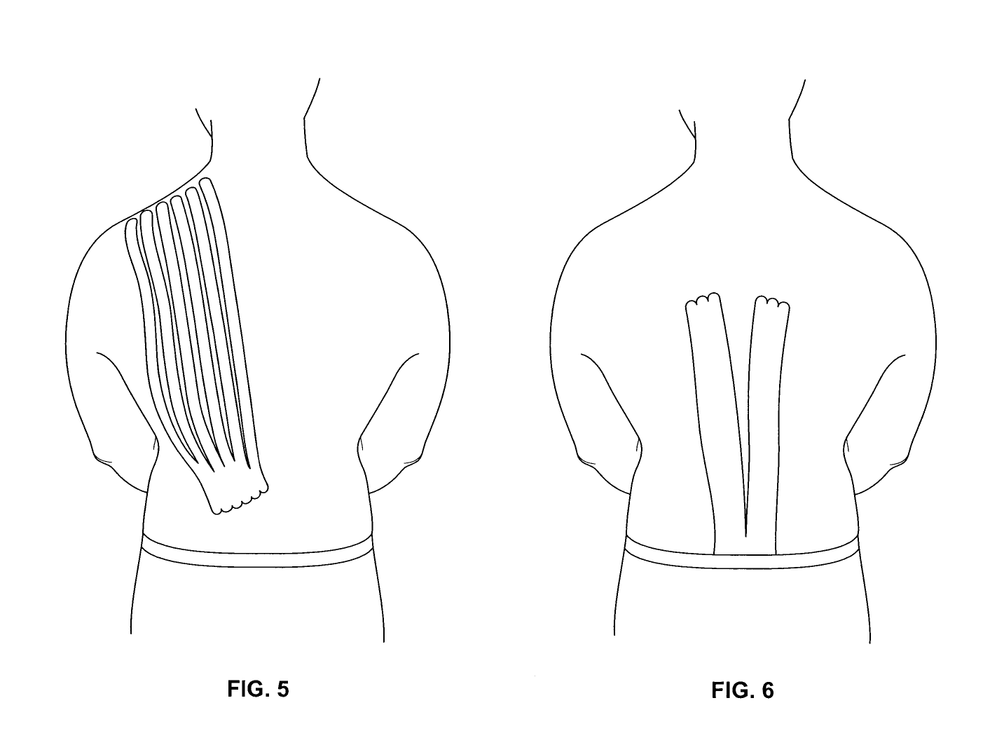

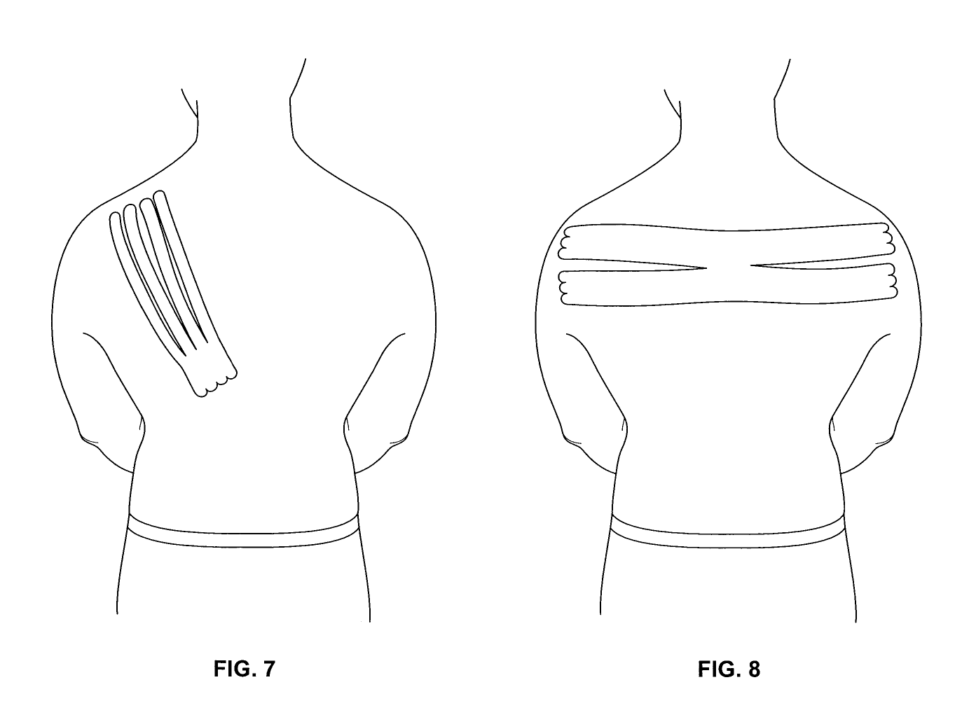

As shown in FIG. 4, the strip may be left uncut and applied in a long/wide application (similar to an I-shape application). Very long/wide lymphatic cuts may be obtained as shown in FIG. 5. A single central longitudinal cut may be made as shown in FIG. 6. As shown in FIG. 7, a shorter lymphatic cut may be obtained by cutting a lymphatic fan shape with shorter finger portions. As shown in FIG. 8, an X-shape application can be formed in a long/wide application.

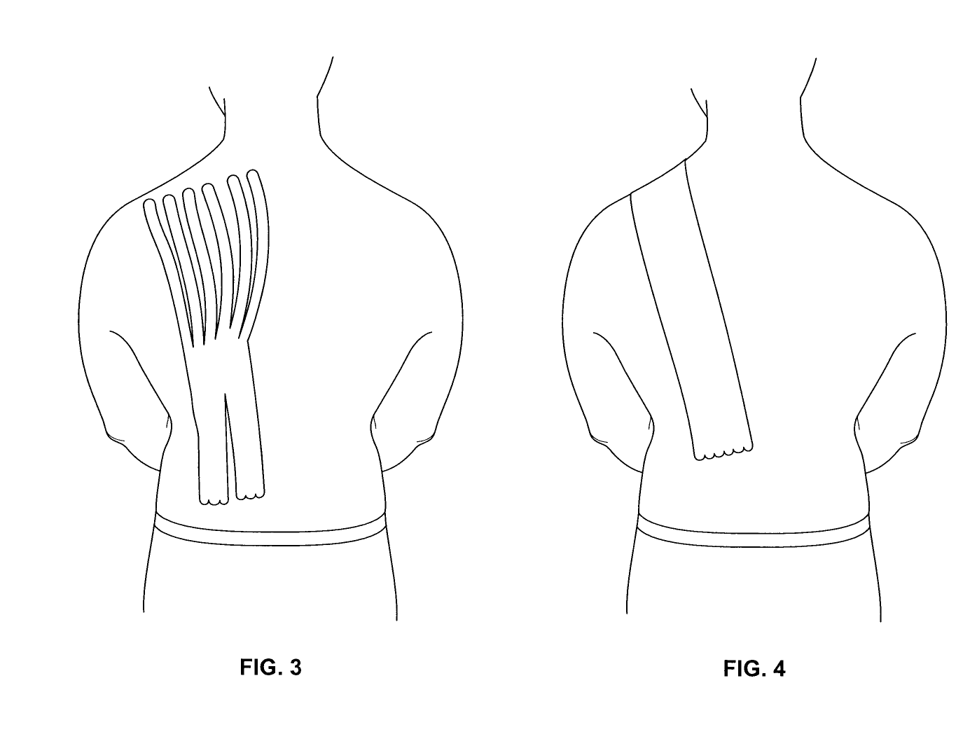

The flexibility provided by the grid lines also permits forming some shapes that are not presently available in precut or roll form. For example, as shown in FIG. 3, the strip may be formed into a hybrid of a fan type strip and a Y-shape type strip. This allows for ease and continuity of application and the ability to combine two (or more) application techniques in one application. In the past, to obtain a shape of this type would have required a combination application of two separate precuts (one fan shape and one Y-shape), with the disadvantages that accompany overlapping precuts.

The user-configurable precut kinesiology tape strip has a number of practical advantages. The strips allow for great flexibility for professional use. There is a lower startup cost since there is no need for the professional user to buy and maintain an inventory of separate applications for each possible treatment. The strips are wider than conventional tape and feature prerounded ends. The grid lines allow even and straight cutting and repeatability. The same shape can be reproduced multiple times for the same patient, and the professional's staff can make up repeat copies in advance. It is also easy to document the shape that was applied, since the grid lines act as a template. The cut strip can be photographed or sketched before application so that its cut grid lines are clearly documented for the patient's file. The clinician may also teach the patient how to cut strips for the patient's own use.

There are also some therapeutic/clinical advantages. Some shapes that were not previously able to be done with single strip can now be done a single strip (including hybrid therapies and large scale applications). There is less of a need for overlapped strips which can delaminate from each other or pull away from the user. The strips allow for therapeutic consistency and documentability, since the grid lines permit accurate shaping and repeatable cutting. Further, the strips allow the professional user to scale a therapy up or down for patients of different sizes (children, larger or bariatric patients), or even animal treatments. Both the length and width are easily and repeatably scalable. There is great flexibility over where to anchor the strip when applying it to a patient. The anchor can be provided at any point along the strip, scalable to the individual case. Longer finger portions can be provided in one direction or the other, and the ends need not be symmetrically disposed. The grid lines also allow for the user to make windows or access gaps or slots for specific purposes (e.g. acupuncture, medicine or fluid delivery). This may allow for integration with medical devices and other interventions.

The scope of the claims should not be limited by the preferred embodiments set forth in the examples, but should be given the broadest purposive construction consistent with the description as a whole.

1. A user-configurable precut kinesiology tape strip for application to a patient, comprising:
a precut strip of kinesiology tape having a first end and a second end;
each of the ends having a plurality of lobes, the lobes being disposed opposite each other in pairs, the lobes on each end being generally adjacent to each other with troughs in between; and
a backing paper over an adhesive side of the precut strip of kinesiology tape, the backing paper being marked with a first set of longitudinal grid lines extending between opposing troughs;
wherein the longitudinal grid lines facilitate cutting of the precut strip of kinesiology tape into any one of a number of possible configurations prior to removal of the backing paper and application to a patient's body.

2. The user-configurable precut kinesiology tape strip of claim 1, further comprising latitudinal grid lines marked at predetermined intervals on the backing paper.

3. The user-configurable precut kinesiology tape strip of claim 2, wherein the latitudinal grid lines have lobes and troughs matching the lobes and troughs of the nearest end of the precut strip of kinesiology tape.

4. The user-configurable precut kinesiology tape strip of claim 1, further comprising a second set of longitudinal grid lines marked on the precut strip of kinesiology tape opposite the first set of longitudinal grid lines on the backing paper.

5. The user-configurable precut kinesiology tape strip of claim 1, wherein the lobes are generally rounded, and the troughs are one of generally V-shaped, or generally notch shaped.

6. (canceled)

7. The user-configurable precut kinesiology tape strip of claim 1, wherein the troughs represent spaces between the lobes.

8. The user-configurable precut kinesiology tape strip of claim 1,
wherein the strip is at least approximately 10 inches in length, and at least approximately 3 inches wide.

9. (canceled)

10. The user-configurable precut kinesiology tape strip of claim 1, wherein each end has at least approximately 3 lobes.

11. A method of configuring a user-configurable precut kinesiology tape strip, the user-configurable precut kinesiology tape strip comprising a precut strip of kinesiology tape having a first end and a second end, and a backing paper over an adhesive side of the precut strip of kinesiology tape, at least one of the backing paper and the precut strip of kinesiology tape being marked with a set of longitudinal grid lines extending between the ends of the precut strip of kinesiology tape; wherein the method comprises the steps of:
cutting along at least a portion of one or more of the longitudinal grid lines to create a plurality of finger portions; and
leaving at least one anchor portion on the precut strip of kinesiology tape, the at least one anchor portion corresponding with a generally uncut portion of the precut strip of kinesiology tape.

12. The method of claim 11, wherein each end has a plurality of lobes, the lobes of opposite ends disposed adjacent each other in pairs, the lobes on each end being generally adjacent each other with troughs therebetween, wherein the grid lines extend between opposing troughs.

13. The method of claim 12, wherein the user-configurable precut kinesiology tape strip further comprises latitudinal grid lines marked at predetermined intervals along either or both of the precut strip of kinesiology tape or the backing paper, and the cutting step further comprises cutting along at least one of the latitudinal grid lines.

14. The method of claim 13, wherein the latitudinal grid lines have latitudinal lobes and troughs matching the lobes and troughs of the nearest end of the strip, and the cutting step further comprises cutting along at least one of the latitudinal grid lines to follow the latitudinal lobes and troughs.

15. The method of claim 13, wherein at least a portion of the longitudinal grid lines are cut prior to cutting at least a portion of the latitudinal grid lines.

16. The method of claim 15, wherein the cutting step comprises cutting only a portion of the latitudinal grid lines to shorten or remove at least one of the finger portions.

17. The method of claim 11, wherein the cutting step comprises cutting along an adjacent pair of the longitudinal grid lines.

18. The method of claim 11, wherein the cutting step comprises cutting along a non-adjacent pair of the longitudinal grid lines.

19. The method of claim 11, wherein the cutting step comprises forming cuts of different lengths.

20. The method of claim 11, wherein the cutting step comprises forming multiple cuts of the same length.

21. The method of claim 11, wherein the cutting step comprises cutting portions of the grid lines generally opposite each other or offset from each other at opposite ends.

22. The method of claim 11, wherein the cutting step is performed by a user.

23. The method of claim 22, wherein the cutting step is performed with scissors.

24. The method of claim 11, further comprising:
applying the anchor portion to the patient; and
applying the finger portions to the patient following the application of the anchor portion.

25. The user-configurable precut kinesiology tape strip claim 1, wherein the longitudinal grid lines extend from one end of the precut kinesiology tape strip to the other and connect the opposing troughs.
